(19) United States
(12) Patent Application Publication
(10) Pub. No.: US 2002/0111377 A1
(43) Pub. Date: Aug. 15, 2002

(54) Transdermal delivery of cannabinoids

(75) Inventor: Audra L. Stinchcomb, Lexington, KY (US)

Correspondence Address: HOFFMAN WARNICK & D'ALESSANDRO, LLC, 3 E-COMM SQUARE, ALBANY, NY, 12207

(73) Assignee: Albany College of Pharmacy, Albany, NY

(21) Appl. No.: 10/032,163

(22) Filed: Dec. 21, 2001

Related U.S. Application Data
(60) Provisional application No. 60/257,557, filed on Dec. 22, 2000.

(51) Int. Cl. A61K 31/352

(52) U.S. Cl. 514/468

(57) ABSTRACT

Disclosed is a method for relieving symptoms associated with illness or discomfort associated with the treatment of illness in a subject. The method includes providing a cannabinoid composition and delivering the cannabinoid transdermally to the subject. The cannabinoid composition used in this method includes at least one cannabinoid selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2. Also disclosed is an occlusive body which includes an impermeable backing and a rate-controlling microporous membrane which together define a cavity in which a cannabinoid is disposed. Methods for increasing the concentration of cannabinoids or cannabinoid metabolites in a subject and for assessing the permeability of skin to cannabinoids are also described.

The present invention claims the benefit of U.S. Provisional Patent Application Serial No. 60/257,557, filed Dec. 22, 2000, which is hereby incorporated by reference.

### FIELD OF THE INVENTION

The present invention is directed to the transdermal delivery of cannabinoids and, more particularly, to methods and materials for transdermally delivering cannabinoids to relieve symptoms associated with illness, to relieve discomfort associated with the treatment of illness, and/or to increase the concentration of cannabinoids and/or cannabinoid metabolites in a subject.

### BACKGROUND OF THE INVENTION

The use of cannabinoids to treat medical illnesses is of great interest to the medical community. Specifically, illnesses such as AIDS and cancer are often accompanied with a lack of appetite. Moreover, patients receiving cancer chemotherapy often experience nausea and vomiting side effects. Chronic pain (especially neuropathic pain), malignant tumors, spasticity (in multiple sclerosis and spinal cord injury), and or dystonia are additional therapeutic targets for cannabinoid therapy. The capability to control or eliminate these problems would greatly increase the quality of life for many patients.

Heretofore, attempts have been made at administering the cannabinoid Δ⁹-THC (Dronabinol) orally, in the form of a capsule. However, severely nauseated patients are often not able to retain the capsule in their stomachs long enough for the drug to take effect. This problem is compounded by the fact that four to six doses of the capsule must be taken around chemotherapy. Another issue with capsules, as well as smoked marijuana, is that patients absorb the drug relatively rapidly and receive high drug concentrations in their body. These high drug concentrations, or peak levels, are often associated with serious psychoactive and other central nervous system side effects.

For these and other reasons, a need remains for alternative methods of delivering cannabinoids to patients. The present invention, in part, is directed to meeting this need.

### SUMMARY OF THE INVENTION

The present invention relates to a method for relieving symptoms associated with illness or discomfort associated with the treatment of illness in a subject. The method includes providing a cannabinoid composition and delivering the cannabinoid transdermally to the subject. The cannabinoid composition used in this method includes at least one cannabinoid selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

The present invention also relates to an occlusive body which includes an impermeable backing and a rate-controlling microporous membrane. The backing and the membrane define a cavity therebetween, and the cannabinoid is disposed within the cavity.

The present invention also relates to a method for increasing the concentration of cannabinoids or cannabinoid metabolites in a subject. The method includes contacting the subject's skin with a compound selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

The present invention also relates to a method for assessing the permeability of skin to a cannabinoid. The method includes providing a skin sample having a first surface and an opposing second surface; providing a donor solution which includes a cannabinoid; and providing a receiver solution which includes from 0.1 to 5% of a polyoxyethylene oleyl ether. The method further includes disposing the skin sample between the donor solution and the receiver solution and detecting cannabinoid present in the receiver solution. In this method, the skin sample is disposed between the donor solution and the receiver solution such that the skin sample separates the donor solution and the receiver solution, such that the donor solution is in contact with the skin sample's first surface, and such that the receiver solution is in contact with the skin sample's second surface.

### BRIEF DESCRIPTION OF THE DRAWINGS

FIG. 1 is a cross-sectional view of an occlusive body in accordance with the present invention.

FIG. 2 is a permeation profile of showing the delivery of a cannabinoid across skin.

FIG. 3 is a bar graph showing permeability of skin samples to a cannabinoid for various receiver solutions.

FIG. 4 is a bar graph showing permeability of skin samples to a cannabinoid for various receiver solutions.

FIG. 5A is a bar graph and FIG. 5B is a permeation profile, each showing permeability of skin samples to a cannabinoid for various receiver solutions.

FIG. 6 is a bar graph showing permeability of skin samples to a cannabinoid for various receiver solutions.

FIG. 7 is a bar graph showing permeability of stripped and intact skin samples to a cannabinoid for various receiver solutions.

FIG. 8 is a bar graph showing maximum flux of a cannabinoid through skin samples for various receiver solutions.

### DETAILED DESCRIPTION OF THE INVENTION

The present invention relates to a method for relieving symptoms associated with illness or discomfort associated with the treatment of illness in a subject.

Subjects which can benefit from the method of the present invention include, for example, mammals, such as humans, particularly humans requiring relief from chronic pain, such as neuropathic pain.

The method of the present invention can be used to relieve the symptoms of a variety of diseases, conditions, syndromes, disorders, and other forms of illness. For example, as explained above, patients suffering from illnesses, such as cancer and AIDS, often experience symptoms, such as lack of appetite, which can be relieved with the method of the present invention. Patients suffering from neuropathy experience chronic pain and other symptoms which can be relieved with the method of the present invention. Patients suffering from multiple sclerosis or spinal cord injury experience spasticity and other symptoms which can be relieved with the method of the present invention. The methods of the present invention can also be used to relieve symptoms associated with dystonia and malignant tumors. The methods of the present invention can also be used to relieve symptoms of stroke, head injuries, neurodegenerative disorders, and other conditions, diseases, and disorders associated with the N-methyl-D-aspartate receptor. The mechanism by which symptoms are relieved is not particularly critical to the practice of the present invention. Illustratively, symptoms can be relieved by directly treating the underlying illness or by blocking the biological pathways by which the illness produces the symptoms.

Moreover, the method of the present invention can be used to relieve discomfort associated with the treatment of illness. Illustratively, the method of the present invention can be used to relieve nausea, vomiting, and/or other discomforts associated with chemotherapy and other treatment regimens used to treat cancer and other illnesses. “Relieve”, as used herein, is meant to include complete elimination as well as any clinically or quantitatively measurable reduction in the subject's symptoms and/or discomfort.

The method of the present invention involves providing a cannabinoid composition. The cannabinoid composition includes at least one cannabinoid selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

“Cannabinoid”, as used herein, is meant to include compounds which interact with the cannabinoid receptor and various cannabinoid mimetics, such as certain tetrahydropyran analogs (e.g., Δ⁹tetrahydrocannabinol, Δ⁸-tetrahydrocannabinol, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ⁶-tetrahydrocannabinol-1,1-dimethylheptyl, (+)-(3S,4S)-7-hydroxy-Δ⁶-tetrahydrocannabinol-1,1-dimethylheptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6,6a,7,8,9,10,10a-octahydro -6-methyl-3-[(R)-1-methyl-4-phenylbutoxy]-1,9-phenanthridinediol 1-acetate)), certain aminoalkylindole analogs (e.g., (R)-(+)-[2,3-dihydro-5-methyl-3-(4-morpholinylmethyl)-pyrrolo[1,2,3-de]-1,4-benzoxazin-6-yl]-1-naphthalenyl-methanone), certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl) -1′,2′,3′,4′,5′,6′-hexahydrobiphenyl), as well as their pharmaceutically acceptable salts, solvates, metabolites (e.g., cutaneous metabolites), and metabolic precursors. Further examples of “cannabinoids” include those compounds described in the references cited below.

“Δ⁹-THC”, as used herein, is meant to refer to Δ⁹-tetrahydrocannabinol as well as to its pharmaceutically acceptable salts, solvates, metabolites (e.g., cutaneous metabolites), and metabolic precursors. Δ⁹tetrahydrocannabinol is marketed under the generic name “dronabinol”.

“Cannabinol”, as used herein, is meant to refer to 6,6,9-trimethyl-3-pentyl-6H-dibenzo[b,d]pyran-1-ol as well as to pharmaceutically acceptable salts, solvates, metabolites (e.g., cutaneous metabolites), and metabolic precursors of 6,6,9-trimethyl-3-pentyl-6Hdibenzo[b,d]pyran-1-ol. The synthesis of 6,6,9-trimethyl-3-pentyl-6H-dibenzo[b,d]pyran-1-ol is described in, for example, Novak et al., Tetrahedron Letters, 23:253 (1982), which is hereby incorporated by reference.

“Cannabidiol”, as used herein, is meant to refer to 2-[3-methyl-6-(1-methylethenyl)-2-cyclohexen-1-yl]-5-pentyl-1,3-benzenediolas well as to pharmaceutically acceptable salts, solvates, metabolites (e.g., cutaneous metabolites), and metabolic precursors of 2-[3-methyl-6-(1-methylethenyl)-2-cyclohexen-1-yl]-5-pentyl-1,3-benzenediol. The synthesis of 2-[3-methyl-6-(1-methylethenyl)-2-cyclohexen-1-yl]-5-pentyl-1,3-benzenediol is described, for example, in Petilka et al., Helv. Chim. Acta, 52:1102 (1969) and in Mechoulam et al., J. Am. Chem. Soc., 87:3273 (1965), which are hereby incorporated by reference.

“Nabilone”, as used herein, is meant to refer to 3-(1,1-dimethylheptyl)-6,6a,7,8,10,10a-hexahydro-1-hydroxy-6,6-dimethyl-9H-dibenzo[b,d]pyran-9-one as well as to pharmaceutically acceptable salts, solvates, metabolites (e.g., cutaneous metabolites), and metabolic precursors of 3-(1,1-dimethylheptyl)-6,6a,7,8,10,10a-hexahydro -1-hydroxy-6,6-dimethyl-9H-dibenzo[b,d]pyran-9-one. 3-(1,1-dimethylheptyl)-6,6a,7,8,10,10a-hexahydro-1-hydroxy-6,6-dimethyl-9H-dibenzo[b,d]pyran-9-one is approved for use in the United Kingdom for treating nausea and vomiting associated with chemotherapy, and its preparation is described, for example, in U.S. Pat. No. 3,968,125 to Archer, which is hereby incorporated by reference.

“Levonantradol”, as used herein, is meant to refer to (−)-(6S,6aR,9R,10aR)-5,6,6a,7,8,9,10,10a-octahydro -6-methyl-3-[(R)-1-methyl-4-phenylbutoxy]-1,9-phenanthridinediol 1-acetate, as well as to pharmaceutically acceptable salts, solvates, metabolites (e.g., cutaneous metabolites), and metabolic precursors of (−)-(6S,6aR,9R,10aR)-5,6,6a,7,8,9,10,10a-octahydro-6-methyl-3-[(R)-1-methyl-4-phenylbutoxy]-1,9-phenanthridinediol 1-acetate. (−)-(6S,6aR,9R,10aR) -5,6,6a,7,8,9,10,10a-octahydro-6-methyl-3-[(R)-1-methyl-4-phenylbutoxy]-1,9-phenanthridinediol 1-acetate is particularly useful in pain control, and its synthesis is described in Belgian Pat. No. 854,655, which is hereby incorporated by reference; in U.S. Pat. Nos. 4,206,225, 4,232,018, and 4,260,764, each to Johnson, which are hereby incorporated by reference; in U.S. Pat. No. 4,235,913 to Johnson et al., which is hereby incorporated by reference; in U.S. Pat. No. 4,243,674 to Bindra, which is hereby incorporated by reference; and in U.S. Pat. Nos. 4,263,438, 4,270,005, and 4,283,569, each to Althuis et al., which are hereby incorporated by reference.

“(−)-HU-210”, as used herein, is meant to refer to (−)-(3S,4S)-7-hydroxy-Δ⁶-tetrahydrocannabinol-1,1-dimethylheptyl as well as to pharmaceutically acceptable salts, solvates, metabolites (e.g., cutaneous metabolites), and metabolic precursors of (−)-(3S,4S)-7-hydroxy-Δ⁶-tetrahydrocannabinol-1,1-dimethylheptyl. (−)-(3S,4S)-7-hydroxy-Δ⁶-tetrahydrocannabinol-1,1-dimethylheptyl is particularly useful in pain control, and its preparation is described in U.S. Pat. No. 4,876,276 to Mechoulam et al. and in U.S. Pat. No. 5,521,215 to Mechoulam et al., which are hereby incorporated by reference.

“(+)-HU-210”, as used herein, is meant to refer to (+)-(3S,4S)-7-hydroxy-Δ⁶-tetrahydrocannabinol-1,1-dimethylheptyl as well as to pharmaceutically acceptable salts, solvates, metabolites (e.g., cutaneous metabolites), and metabolic precursors of (+)-(3S,4S)-7-hydroxy-Δ⁶-tetrahydrocannabinol-1,1-dimethylheptyl. (+)-(3S,4S)-7-hydroxy-Δ⁶-tetrahydrocannabinol-1,1-dimethylheptyl is sometimes referred to as HU-211 and/or dexanabinol; it is an antagonist of the N-methyl-D-aspartate receptor; and its preparation is described in U.S. Pat. No. 4,876,276 to Mechoulam et al. and in U.S. Pat. No. 5,521,215 to Mechoulam et al., which are hereby incorporated by reference.

“11-hydroxy-Δ⁹-THC”, as used herein is meant to refer to 11-hydroxy-Δ⁹-tetrahydrocannabinol as well as to its pharmaceutically acceptable salts, solvates, metabolites (e.g., cutaneous metabolites), and metabolic precursors. 11-hydroxy-Δ⁹-tetrahydrocannabinol is a more hydrophilic, psychoactive metabolite of Δ⁹-tetrahydrocannabinol, and its laboratory synthesis has been described in Siegel et al., J. Org. Chem., 54:5428 (1989), which is hereby incorporated by reference.

“Δ⁸-THC-11-oic acid”, as used herein, is meant to refer to Δ⁸-tetrahydrocannabinol-11-oic acid, as well as to its pharmaceutically acceptable salts, solvates, metabolites (e.g., cutaneous metabolites), and metabolic precursors. Δ⁸-tetrahydrocannabinol-11-oic acid is a naturally occurring derivative of 6a,7,10,10a-tetrahydro-6,6,9-trimethyl-3-pentyl-6H-dibenzo[b,d]pyran-1-ol (which is a minor component of Cannabis sativa) and is produced from 6a,7,10,10a-tetrahydro-6,6,9-trimethyl-3-pentyl-6H-dibenzo[b,d]pyran-1-ol via a series of biotransformations mediated primarily by mammalian liver enzymes. Δ⁸-tetrahydrocannabinol-11-oic acid can also be produced synthetically by reference to the synthetic schemes set forth in U.S. Pat. No. 6,162,829 to Burstein, which is hereby incorporated by reference. Δ⁸-tetrahydrocannabinol -11-oic acid is more hydrophilic than 6a,7,10,10a-tetrahydro -6,6,9-trimethyl-3-pentyl-6H-dibenzo[b,d]pyran -1-ol, and it has analgesic activity.

“CP 55,940”, as used herein, refers to 4-(1,1-dimethylheptyl)-2,3′-dihydroxy-6′alpha-(3-hydroxypropyl)-1′,2′,3′,4′,5′,6′-hexahydrobiphenyl, as well as to its pharmaceutically acceptable salts, solvates, metabolites (e.g., cutaneous metabolites), and metabolic precursors. 4-(1,1-dimethylheptyl)-2,3′-dihydroxy-6′alpha-(3-hydroxypropyl)-1′,2′,3′,4′,5′,6′-hexahydrobiphenyl is sometimes referred to as (−)-cis-3-[2-Hydroxy-4-(1,1-dimethylheptyl)phenyl]-trans-4-(3-hydroxypropyl) cyclohexanol, and it is commercially available from Tocris Cookson, Inc., Ellisville, Mo. Its preparation has been described in U.S. Pat. No. 4,371,720 to Johnson et al. and U.S. Pat. No. 4,663,474 to Urban, which are hereby incorporated by reference.

“R(+)-WIN 55,212-2”, as used herein, refers to (R)-(+)-[2,3-dihydro-5-methyl-3-(4-morpholinylmethyl)-pyrrolo[1,2,3-de]-1,4-benzoxazin-6-yl]-1-naphthalenyl-methanone, as well as to its pharmaceutically acceptable salts, solvates, metabolites (e.g., cutaneous metabolites), and metabolic precursors. (R)-(+)-[2,3-dihydro-5-methyl-3-(4-morpholinylmethyl)-pyrrolo[1,2,3-de]-1,4-benzoxazin-6-yl]-1-naphthalenyl-methanone (in its mesylate form) is commercially available, for example, from Tocris Cookson, Inc., Ellisville, Mo., and from Research Biochemicals International, Natick, Mass.

The cannabinoid composition can further include one or more additional cannabinoids. The one or more additional cannabinoids can be selected from the aforementioned list of cannabinoids or it (they) can be selected from cannabinoids which are not contained in the aforementioned list, such as Δ⁸-THC, high affinity cannabinoid receptor agonists (other than R(+)-WIN 55,212-2 and CP 55,940), and the like. Illustratively, the cannabinoid composition can include two or more cannabinoids, each being selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

“Metabolic precursors” of cannabinoids, as used herein, are meant to include prodrugs and other materials that are metabolized in the subject's body (e.g., cutaneously or systemically or both) to a cannabinoid or an active cannabinoid mimetic. Suitable metabolic precursors include those that are less lipophilic (i.e., more water soluble) relative to the cannabinoid into which they are metabolized. Examples of such metabolic precursors include those described in, for example, U.S. Pat. No. 5,847,128 to Martin et al., which is hereby incorporated by reference.

“Metabolites” of cannabinoids, as used herein, are meant to include compounds which are produced by the metabolic processes (e.g., cutaneous metabolic processes and/or systemic metabolic processes) of the subject's body. Suitable metabolites can be identified, for example, by studying the kinetics of drug enzymatic metabolism in skin homogenates. Illustratively, skin homogenates can be prepared from 250-μm dermatomed fresh healthy abdominal plastic surgery samples. The skin is homogenized (e.g., using a Polytron tissue homogenizer and ground glass homogenizer fitted with a glass pestle) in 4-(2-hydroxyethyl)-1-piperazineethanesulfonic acid (“HEPES”)-buffered Hanks' balanced salt solution. Whole homogenates can be used for these studies or, if significant mitochondrial or nuclear metabolism is found not to occur (e.g., by comparing the degree of metabolism in the supernatant the degree of metabolism in the whole homogenate), the studies can be carried out on only the supernatant fraction. The drug (solubilized in, for example, buffer, ethanol, dimethylsulfoxide, or combinations thereof) is then incubated with the homogenate (or supernatant) along with NADPH (or a generating system), NADH, MgCl₂, and bovine serum albumin. The total volume of ethanol in the reaction mixture should be small (e.g., under 2%) to help minimize ethanol's detrimental effects on the enzymes. After incubating for a period of time, the reaction is terminated with 15% trichloroacetic acid, and the drug and its metabolites are obtained by solid-phase extraction. The metabolite or metabolites formed can then be identified and assayed by any suitable method (e.g., HPLC).

As one skilled in the art will recognize, optimization of the method of the present invention will involve consideration of a variety of factors in selecting the cannabinoid to be used. One such factor is skin permeability. Several physicochemical factors influence the ability of cannabinoids to penetrate the skin. These include the cannabinoid's molecular weight, its molecular volume, its lipophilicity, its hydrogen bonding potentials, its polarity, etc.

As indicated above, once the cannabinoid composition is provided, the cannabinoid is delivered transdermally to the subject, for example, by iontophoresis; by phonophoresis; by using microneedle technologies; by applying the cannabinoid as a topical cream, salve, ointment, or other topical formulation; and/or by using delivery devices such as bandages, patches, and/or the like. Generally speaking, transdermal delivery involves contacting the cannabinoid composition with the subject's skin under conditions effective for at least one of the provided cannabinoids to penetrate the skin.

Illustratively, the cannabinoid composition can be formulated as a topical cream, salve, or ointment. The topical formulations can include inert diluents and carriers as well as other conventional excipients, such as wetting agents, preservatives, and suspending and dispersing agents. In addition to the above, generally non-active components, topical formulations containing the cannabinoid composition can further include other active materials, particularly, active materials which have been identified as useful in the treatment of pain, discomfort, or other conditions associated with a subject's illness and which can usefully be delivered transdermally to the subject. For instance, such other active materials can include analgesics, such as opiates and other analgesic active materials which operate on non-cannabinoid receptors. Where, for example, opiates are included, transdermally deliverable opiates are particularly preferred. One example of a transdermally deliverable opiate is fentanyl. The topical formulation can be applied directly to the skin and then optionally covered (e.g., with a bandage of gauze) to minimize the likelihood of its being disturbed. Alternatively, the topical formulation can be coated on the surface of a bandage, gauze, etc., and the bandage, gauze, etc. can then be applied to the skin of the subject such that the topical formulation is in direct contact with the subject's skin.

Alternatively, the cannabinoid can be delivered transdermally to the subject by formulating the cannabinoid composition into a bandage, pad, or other type of patch which can be applied to the subject's skin.

Illustratively, matrix-type transdermal patches, in which the selected cannabinoid is disposed in an adhesive matrix, can be employed. The matrix-type transdermal patch can further include other cannabinoids and other active materials (e.g., analgesics, such as opiates) for transdermal delivery to the subject with the selected cannabinoid. Suitable adhesives for use in such matrix-type transdermal patches include polyisobutylenes, acrylates, silicone, and combinations thereof.

Still other patches suitable for use in the practice of the present invention include those described in U.S. Pat. 5,223,262 to Kim et al., which is hereby incorporated by reference.

In another illustrative embodiment, the bandage, pad, or other type of patch can be one which is capable of controlling the release of the cannabinoid such that transdermal delivery of the cannabinoid to the subject is substantially uniform and sustained over a period of at least 12 hours, such as at least 24 hours, at least 48 hours, and/or at least 4 days. Such a bandage, pad, or other type of patch which can be used in the practice of the method of the present invention can take the form of an occlusive body, such as the occlusive body described below. In practice, the occlusive body which includes the cannabinoid is positioned on the subject's skin under conditions effective to transdermally deliver the selected cannabinoid to the subject's skin. Such conditions can include, for example, positioning the occlusive body on a portion of the subject's skin which is not covered with hair; where necessary, shaving the hair from the selected portion of the subject's skin; and/or orienting the occlusive body on the skin such that the cannabinoid, when released from the occlusive body, contacts the subject's skin.

In another aspect thereof, the present invention, relates to an occlusive body which includes a cannabinoid; an impermeable backing; and a ratecontrolling microporous membrane. The backing and the membrane define a cavity therebetween, and the cannabinoid is disposed within this cavity.

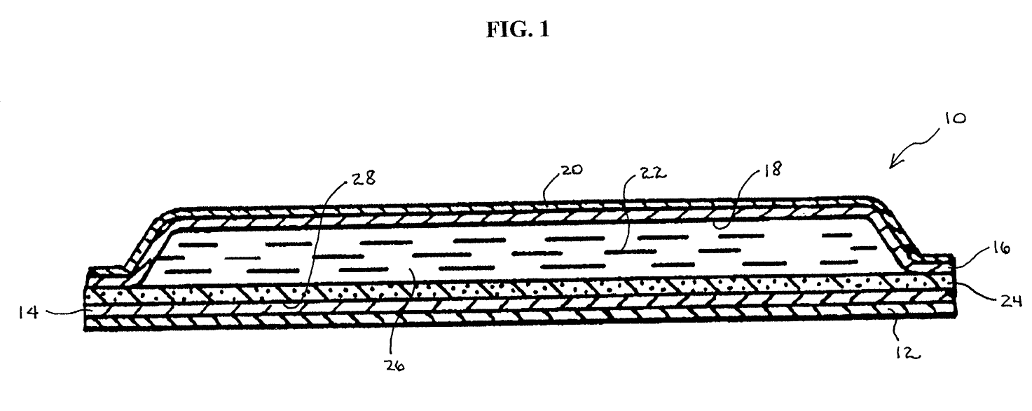

An occlusive body in accordance with the present invention and that is suitable for use in the practice of the method of the present invention is illustrated in FIG. 1.

Referring now to FIG. 1, there is illustrated occlusive body 10. Occlusive body 10 includes impermeable backing 16 having optional polyester face 18, cannabinoid composition 22, and rate-controlling microporous membrane 24. Rate-controlling microporous membrane 24 is shown heat-sealed around the periphery of its upper face to optional polyester face 18 of impermeable backing 16. However, other methods of sealing rate-controlling microporous membrane 24 to impermeable backing 16 (or to optional polyester face 18 of impermeable backing 16) can be employed. Impermeable backing 16 is illustrated as including optional aluminized layer 20 on the outer face thereof. Impermeable backing 16 and rate-controlling microporous membrane 24 define cavity 26, and cannabinoid composition 22 is disposed in cavity 26. Over time, the cannabinoid contained in cannabinoid composition 22 permeates through rate-controlling microporous membrane 24 and optional adhesive layer 14, which is illustrated as being coated on the lower face of rate-controlling microporous membrane 24.

As indicated above, cannabinoid composition 22 contains at least one cannabinoid. Cannabinoids for use in the occlusive body of the present invention can be selected from the group consisting of Δ⁹-THC, Δ⁸-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2. Preferably, cannabinoids for use in the occlusive body of the present invention are selected from the group consisting of cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2. Mixtures of these and other cannabinoids can also be employed. Preferably, the cannabinoid is selected such that it is sufficiently hydrophobic to pass through rate-controlling microporous membrane 24. In addition, cannabinoid composition 22 can also include other inert or active materials, such as those discussed above with regard to topical formulations and/or such as those described below.

Cannabinoid composition 22 can include an aqueous medium, which can contain a water- and oil-miscible solvent. The cannabinoid composition can also contain a material which enhances the cannabinoid's permeation of the skin. Depending on the nature of the chosen solvent, the solvent can also act as the permeation enhancer, or a separate permeation enhancer having the desired miscibility can be added to the cannabinoid composition. Illustrative permeation enhancers that can be used in the occlusive bodies of the present invention include ethanol and oleic acid. Preferably the cannabinoid is present together with at least one diluent so that the cannabinoid accounts for no more than about 25% by weight of the contents of the occlusive body's cavity.

The cannabinoid composition can also include one or more inhibitors of cannabinoid metabolism, particularly in cases where inhibition of cutaneous metabolism is needed to increase therapeutic drug levels. Such inhibitors of cannabinoid metabolism can include inhibitors of the P450 enzymes or other identified critical enzymatic processes. Suitable inhibitors of cannabinoid metabolism include, for example, essential oils which inhibit the activity of cytochrome P450 3A in the skin, such as those described in U.S. Pat. No. 5,716,928 to Benet et al., which is hereby incorporated by reference. Some of these essential oils may also act as transdermal penetration enhancers, thus providing a dual mechanism of percutaneous penetration increase.

Rate-controlling microporous membrane 24 can optionally be made of a single-ply material, or it can be made of a multi-ply material. Only the inner layer of such a membrane needs to be hydrophobic (in the case that the cavity contents are hydrophilic) or hydrophilic (in the case that the cavity contents are hydrophobic). Thus, in one embodiment, an additional permeable membrane is in contact with exterior surface 28 of ratecontrolling microporous membrane 24, and the additional permeable membrane has wetting properties which are the same as, or different from the wetting properties of rate-controlling microporous membrane 24.

It is believed that the greater the difference in wetting properties between the cavity contents and rate-controlling microporous membrane 24 (or the innermost layer of rate-controlling microporous membrane 24 if a multi-ply membrane is used), the wider the range of useful solvents and the more linear the release of the drug. Accordingly, it is desirable to employ either a strongly hydrophobic or a strongly hydrophilic ratecontrolling microporous membrane 24 (or the innermost layer of the rate-controlling microporous membrane 24 if a multi-ply membrane is used) in conjunction with strongly hydrophilic cavity contents and strongly hydrophobic cavity contents, respectively. It should be noted that the cavity contents can be made hydrophilic by adding surface-active agent, such as an anionic surface-active agent (e.g., sodium lauryl sulphonate), a cationic surface active agent (e.g., cetrimide), or a non-ionic surface active agent (e.g., TWEEN 20®).

Occlusive body 10 can also have an outer layer of an impervious material, such as a layer of aluminum foil or other metal or plastic laminate, to prevent seepage or leaching of the contents of the cavity 26. The cavity side of rate-controlling microporous membrane 24 can be faced with an area-reducing mesh formed, for example, from a non-woven fabric or from a perforated impermeable material such as aluminum foil.

Rate-controlling microporous membrane 24 can be made of any suitable membrane material, such as a hydrophobic and microporous membrane material, for example, CELGARD® 2500 polypropylene of thickness 0.025 mm (1 mil) and pore size 0.4-0.04 microns.

Exterior surface 28 of rate-controlling microporous membrane 24 (i.e., the face distant from cavity 26) can optionally be coated with adhesive layer 14, for example, having a thickness of, for example, about 30 micrometers. Any suitable dermatologically acceptable pressure sensitive adhesive that does not react chemically with the cavity contents or prevent passage of the cannabinoid through the membrane from being rate-controlling can be used for adhesive layer 14. Thus, the adhesive can be chosen such that the cannabinoid passes reasonably rapidly through adhesive layer 14, though some retardation is acceptable in practice. The adhesive can be, for example, an elastomeric silicone polymer. Other suitable adhesives include polyisobutylenes and acrylates. Optional release liner 12, such as a sheet of release coated paper or other material, can be used to cover adhesive layer 14 until the occlusive body 10 is to be used, thus preventing cannabinoid permeation prior to contacting occlusive body 10 with the subject's skin. Immediately prior to use, release liner 12 is stripped from adhesive layer 14, and occlusive body 10 is adhered to the subject's skin (e.g., of the arm) (not shown) by the exposed adhesive layer 14.

It is to be understood that adhesive layer 14 is but one of many suitable ways for attaching the occlusive body 10 to the subject's skin. For example, as an alternative to using adhesive layer 14, a separate tape or bandage material can be employed to attach the occlusive body of the present invention to the subject's skin.

The occlusive body of the present invention can further include a viscous flowable gel which is disposed within the occlusive body's cavity and which immobilizes the cannabinoid within the cavity. Such gel formulations can be useful to reduce the likelihood of abrupt absorption of the cannabinoid in the event of sudden rupture of the cavity and release of the cavity contents onto the skin. Suitable gel formulations can be achieved by making the viscosity of the cavity contents sufficiently high such that they are resistant to spreading in the event of cavity puncture. Illustratively, methyl cellulose in water can be used as a viscosity modifier in such gel formulations. In certain situations, the use of methyl cellulose in combination with the cannabinoid composition can also be advantageous in that the methyl cellulose can also function as a surface active agent to enhance the hydrophilicity of the cavity contents.

The cannabinoid may be mixed with up to 2% (typically about 1% by weight) of oil of Melaleuca Alterniifolia (Tea Tree Oil) or another bactericide before being introduced into the cavity. Tea Tree Oil or another bactericide can also be mixed with an adhesive to form a layer covering a face of the rate-controlling microporous membrane remote from the cavity. The major constituents of Tea Tree Oil are 1-terpinen-4-ol and terpinene with minor amounts of 1,8-cineole and p-cymene, and its properties, together with those of other Australian essential oils, are described in Beylier, Perfumer & Flavorist, 4:23 (April/May 1979), which is hereby incorporated by reference. Tea Tree Oil may be substituted by other essential oils that possess antibacterial qualities. Preferably the Tea Tree Oil is present in an amount of from 0.05% to 2% by weight of the liquid contents of the cavity.

In this invention, the rate-controlling microporous membrane can be a hydrophobic microporous material, such as hydrophobic microporous polypropylene or polyethylene. The cavity contents can illustratively include, in addition to the cannabinoid, a wetting agent water based gel formed, for example, using methyl cellulose. As a further illustration of an occlusive body of the present invention, the rate-controlling microporous membrane can be a hydrophobic microporous polypropylene membrane and the cavity can contain, in addition to the cannabinoid, a water-based gel containing an amount of methyl cellulose (e.g., 5%) which gives a linear release of the cannabinoid while retaining water and solids. It may be expedient to dissolve the cannabinoid in an appropriate pharmaceutically acceptable vehicle, which will carry the active substance through the rate-controlling microporous membrane. Moreover, the rate of delivery of the cannabinoid through the rate-controlling microporous membrane into the blood stream of the subject can be varied by varying the surface area, thickness, and composition of the membrane; by varying the weight ratio of cannabinoid-to-vehicle; and by varying the hydrophilicity of the cavity contents.

In this manner, the dosage rate can be varied over a wide range by suitable adjustment of various parameters of the occlusive body, while maintaining a substantially uniform dosage rate. However, in order to minimize variations in dosage rate between different patients owing to variations in their skin resistance, the permeability of the rate-controlling microporous membrane is preferably less than (e.g., from 0.75 to 0.9 times) the permeability of the least permeable skin likely to be encountered in the use of the occlusive body.

Further details with regard to the construction and configuration of occlusive bodies suitable for use in the practice of the present invention can be found, for example, in U.S. Pat. No. 5,254,346 to Tucker et al., which is hereby incorporated by reference. It should be understood that, in addition to the aforementioned cannabinoid or combination of cannabinoids, other active materials, such as opiates and other analgesics, can be contained in the occlusive body's cavity and delivered transdermally through the rate-controlling microporous membrane together with the cannabinoid or cannabinoid combination.

The present invention, in yet another aspect thereof, relates to a method for assessing the permeability of skin to cannabinoids, particularly lipophilic cannabinoids.

The method includes providing a skin sample. Suitable skin samples can be obtained, for example from abdominal plastic surgery procedures. Typically, the sample is dermatomed to a thickness of from 150-600 μm, preferably from 200-300 μm, more preferably around 250 μm. The dermatomed skin sample is generally substantially planar and has a first surface and an opposing second surface.

The method further includes providing a donor solution which includes the cannabinoid to be studied dissolved in a suitable vehicle. Preferably, the vehicle is chosen such that a substantial concentration is achieved and such that, at this substantial concentration, the cannabinoid forms a near (e.g., greater than 80%) saturated solution.

The method further includes providing a receiver solution which includes from 0.1 to 5% of a polyoxyethylene oleyl ether. Suitable polyoxyethylene oleyl ethers include polyoxyethylene 20 oleyl ether, e.g., BRIJ® 98, which is available from Sigma (St. Louis, Mo.). Illustratively, the concentration of polyoxyethylene 20 oleyl ether can be from about 0.1 to about 5%, such as from about 0.2 to about 4%, from about 0.2 to about 3%, from about 0.3 to about 3%, from about 0.4 to about 3%, from about 0.5 to about 3%, from about 0.5 to about 5%, and/or about 0.5%.

In the practice of the method of the present invention, the skin sample is disposed between the donor solution and the receiver solution such that the skin sample separates the donor solution and the receiver solution, such that the donor solution is in contact with the skin sample's first surface, and such that the receiver solution is in contact with the skin sample's second surface. Preferably, the skin sample is arranged such that the epidermal side of the skin sample is in contact with the donor solution. The arrangement of donor solution/skin sample/receiver solution can be held in place using any suitable apparatus. One suitable commercially available apparatus is the PermeGear In-Line Diffusion Cell, which is available from PermeGear, Inc. (Riegelsville, Pa.). Typically the donor solution is permitted to remain in contact with the skin sample for a period of time ranging from several minutes to several weeks (e.g., 2-4 days), during which time some portion of the cannabinoid permeates through the skin sample and into the receptor solution. The method further includes detecting cannabinoid present in the receiver solution, such as by chromatography (e.g., HPLC). The method can further include quantifying the amount of cannabinoid present in the receiver solution, calculating permeability rates, lag times, and other such useful information, as described further below.

The present invention is further illustrated with the following examples.

### EXAMPLES

### Example 1

### Delivery of WIN 55,212-2 Across Skin

WIN 55,212-2 Mesylate (melting point 244-245° C.) was purchased from Research Biochemicals International, Natick, Mass. Reagent-grade chemicals were used as received.

The skin permeation study was carried out using skin excised during abdominal reduction surgery. The skin sample was harvested from the abdomen using a Padgett dermatome set to 250 μm; the skin sample was frozen at −20° C. for one week. The frozen skin sample was thawed and used at the 250 μm split-thickness for the diffusion study. Three PermeGear In-Line Flow Cells were used for the skin permeation study. The receiver fluid was 6% BRIJ 98 (Polyoxyethylene 20 Oleyl Ether), in order to increase the partitioning of this extremely lipophilic drug into the receiver. The receiver fluid was pumped through the diffusion cells at a rate of one milliliter per hour. The receiver samples were refrigerated until HPLC analysis. The temperature of the diffusion cells was maintained at 32° C. with a circulating water bath. The diffusion experiment was initiated by charging the donor compartment with 0.25 ml of WIN 55,212-2 Mesylate in propylene glycol (50 mg/ml). Water was not used as the drug vehicle, in order to prevent the low water solubility of WIN 55,212-2 from significantly influencing the diffusion rate.

Samples were analyzed using a HPLC system which consisted of a Waters 717 Autosampler, 501 Pumps, and a 484 Tunable UV Absorbance Detector with Millennium Chromatography Software. A reversed phase Beckman 5 μm particle 4.6 mm×25 cm C-18 column was used with the UV detector set at a wavelength of 215 nm. The mobile phase consisted of 0.05 M monobasic potassium phosphate:acetonitrile (300 ml:700 ml) at a flow rate of 2 ml/min. The sensitivity of the assay was 10 ng/ml, and the WIN 55,212-2 had a retention time of 3.4 minutes. Standard curves exhibited excellent linearity over the entire concentration range employed in the assays.

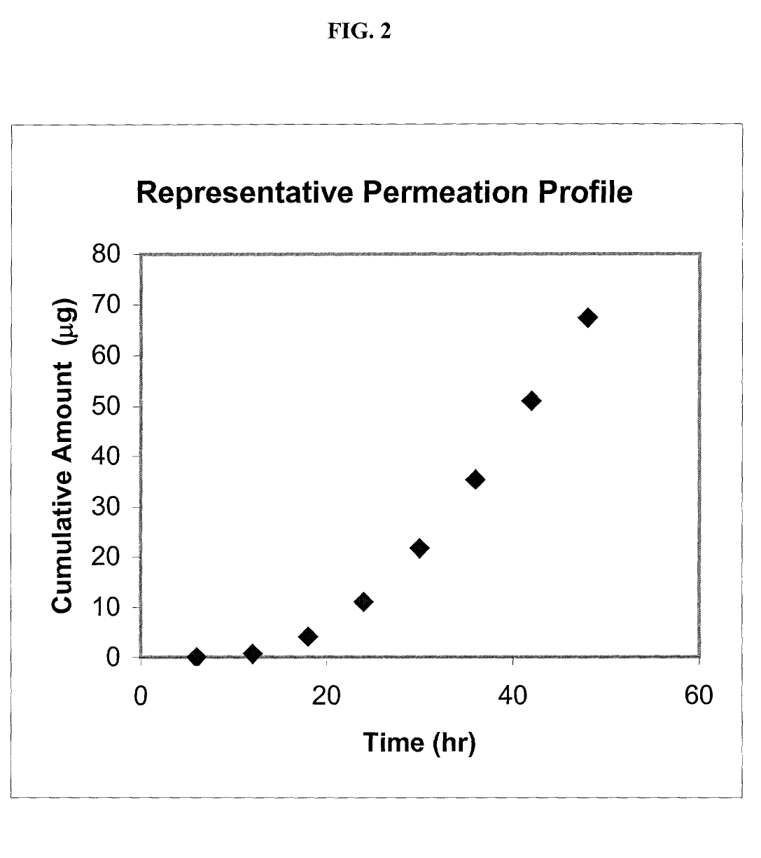

Skin permeation data were plotted as the cumulative amount of drug collected in the receiver compartment as a function of time. The lag times and steady-state fluxes were calculated using the terminal, linear portions of these curves. The slopes (Jₛ) through these curves were determined using linear regression analysis. In all cases, the coefficients of determination for the lines were >0.99. A representative permeation profile is shown in FIG. 2.

Lag times were determined by extrapolating the steady-state curves to the X-axis. The permeability coefficient was calculated from Fick's law of diffusion:

( )(dM/dt)=J_{s=PΔC}

where Jₛ is the flux in the steady state (e.g., in micrograms per cm² per hour); A is the area of the membrane, 0.95 cm²; P is the effective permeability coefficient (e.g., in centimeters per hour); and ΔC is the concentration difference between the diffusion cell's chambers. Since build up in the receiver cell was kept to a minimum throughout the studies, the ΔC is well approximated by the donor concentration.

The skin permeation experiment was run for 48 hours, during which time a steady-state flux was obtained. The diffusion lag time for these experiments averaged to be 24 hours. Although the donor compartment did not contain a saturated drug solution, the drug depletion from the donor compartment was minimal (<1%). The mean flux of these diffusion experiments was found to be 2.6 (±1.1 standard deviation, n=3) μg/cm²/h. The effective permeability coefficient, P, was calculated using Fick's law of diffusion as 1.4×10⁻⁸ cm/s.

These diffusion values are similar in magnitude to the reported full-thickness human skin Δ⁸-THC flux rate of 3.5 μg/cm²/hr, and the permeability coefficient of 3.6×10⁻⁸ cm/s reported in Touitou et al., Int. J. Pharm., 43:9-15 (1988), which is hereby incorporated by reference. The WIN 55,212-2 flux would have been a higher value if enough drug had been available to apply a saturated solution to the skin. One of the practical goals elucidated from this skin permeation study is to choose a vehicle that solubilizes enough drug, but not so much drug that making a saturated solution becomes excessively expensive.

### Example 2

### Optimization of in vitro Experimental Conditions for Studying Permeation Through and Metabolism of Cannabinoids by Human Skin

The permeability of human skin has been studied for several decades. The skin consists of two major layers, the outer epidermis and the inner dermis. The stratum corneum (“SC”), the outermost 10-20 μm of the epidermis, is responsible for the skin's excellent diffusional resistance to the transdermal delivery of most drugs. Most of the skin's enzymatic activity lies in the basal cell layer of the viable epidermis. Fibrous collagen is the main structural component of the dermis. The skin vasculature is supported by this collagen and lies a few microns underneath the epidermis. Basically, it is here that permeation ends and systemic uptake begins. Many researchers have developed skin permeability relationships based on the physicochemical parameters (molecular weight, molecular volume, lipophilicity, hydrogen-bonding potentials, polarity, etc.) of skin penetrants. However, when dealing with transdermal administration of cannabinoids, these skin permeability relationships need to be modified to take into account the potential complications of extreme lipophilicity and concurrent metabolism of these drugs.

Generally speaking, cutaneous metabolism of transdermally delivered drugs is a potential pitfall to therapeutic success. For example, transdermally delivered testosterone and estradiol undergo significant cutaneous metabolism. However, as discussed further below, cutaneous metabolism of cannabinoids can be exploited when designing transdermal prodrugs or when delivering drugs that are converted to active metabolites. For example, it is likely that cannabinoids undergo significant metabolism as they diffuse through viable human skin. The low oral bioavailability of dronabinol (0.1-0.2) is an indication that extensive metabolism may occur; however, some compounds that undergo extensive systemic metabolism do not undergo biotransformation during transit through the skin (Collier et al., “Cutaneous Metabolism,” pp. 67-83 in Bronaugh et al., eds., In Vitro Percutaneous Absorption: Principles, Fundamentals, and Applications, Boca Raton, Fla.: CRC Press (1991), which is hereby incorporated by reference). Most enzymes in the skin have 1-10 percent of the enzymatic specific activity they have in the liver, although other enzymes have equivalent specific activity in both organs. Oxidation generates the main metabolites of Δ⁸-THC and Δ⁹-THC, by aliphatic hydroxylation at the eleven-position carbon, further oxidation to an 11-oic acid, and subsequent glucuronidation. Nabilone forms a diol by reduction at the 9-keto group. Levonantradol is rapidly deacetylated. Evidence suggests that CP 55,940 may undergo side chain hydroxylation.

Selection and optimization of cannabinoids for transdermal delivery requires an understanding of their cutaneous metabolism. Furthermore, since skin metabolism of topical in vivo studies cannot easily be distinguished from blood, liver, or other tissue metabolism, cutaneous metabolism is better studied in vitro. However, the success of any such in vitro study depends heavily on finding ideal conditions to simulate in vivo conditions, especially in maintaining tissue viability. Thus, selection of an optimal receiver solution is critical to the success of any such in vitro studies.

Therefore, applicant has undertaken a study to optimize in vitro experimental conditions for the measurement of Δ⁹-tetrahydrocannabinol across human skin. Additionally, intact and stripped skin were also compared in order to determine if the SC provided significant resistance to the diffusion of highly lipophilic Δ⁹tetrahydrocannabinol. The study and results are described below.

The following materials were used in this study. Δ⁹-tetrahydrocannabinol in 95% ethyl alcohol was obtained in ampules from National Institute of Drug Abuse (Research Triangle Park, N.C.). Hanks balanced salts modified powder, Bovine Albumin Fraction V (“BSA”), potassium phosphate monobasic anhydrous, sodium bicarbonate, and Polyoxyethylene 20 Oleyl Ether (BRIJ 98®) were obtained from Sigma (St. Louis, Mo.). Propylene glycol, 4-(2-hydroxyethyl)-1-piperazineethanesulfonic acid (“HEPES”), triethylamine (“TEA”), gentamycine sulfate, acetonitrile (HPLC grade) and 20 ml Scintillation vials were obtained from Fisher Scientific (Fairlawn, N.J.).

The following instruments and accessories were used in this study. PermeGear flow-through diffusion cells having a surface area of 0.95 cm² were obtained from PermeGear (Hellertown, N.J.). Water Bath 280 series and Shallow Form Shaker Bath were obtained from Precision (Winchester, Va.). Isotemp 2006S water circulator was obtained from Fisher Scientific (Fairlawn, N.J.). Retriever IV fraction collector was obtained from ISCO Inc. (Lincoln, Nebr.). PUMPPRO® MPL static pump was obtained from Watson Marlow (Wilmington, Mass.). Sartoris BP211D model balance was obtained from Sartoris (Edgewood, N.Y.). Padgett Dermatome was obtained from Padgett Instruments (Kansas City, Mo.). HPLC with autosampler (200 series model) and UV detector 785A were obtained from Perkin Elmer. Autosampler vials, nonsilanized/silanized were obtained from Kimble Glass (Vineland, N.J.). All glassware employed in the study was sterilized with 70% v/v ethanol/water.

Receiver solutions containing Hanks-HEPES balanced salt solution (“HHBSS”) and BSA were prepared as follows. HHBSS was prepared by dissolving 9.8 g Hanks balanced salt mixture along with 5.96 g of HEPES and 0.35 g of sodium bicarbonate in 1000 ml of Mili-Q distilled water. The pH was adjusted to 7.4 with 1 N HCl or 1 N NaOH, the solution was filtered through a Milipore filtration system using a 0.2 μm membrane, and 50 mg of gentamycin was added to minimize microbial contamination. Appropriate amounts of BSA, either 4% or 6% (based on the experimental design), were then dissolved into the resulting solutions to produce the HHBSS/BSA receiver solutions.

Receiver solutions containing BRIJ 98 were prepared by dissolving appropriate amounts of BRIJ 98 (0.5% and 6% w/v) in 1000 ml of Mili-Q distilled water.

Human skin samples were prepared as follows. Skin tissue samples from patients having undergone abdominoplasty were obtained from National Cancer Institute Cooperative Human Skin Tissue Network. The samples were dermatomed immediately upon arrival (usually less than 24 hr after harvesting) to obtain 250 μm intact skin samples. The samples were either used immediately or wrapped and then frozen at −20° C. The required fresh tissue portions for immediate use were sliced according to diffusion cell disk area. The removed portions were transferred immediately onto diffusion cell disks that had been previously sterilized with 70% v/v ethanol/water, making sure that the dermal portions were exposed towards the receiver solution. The disks were fixed onto their holders with the aid to hold the tissue firmly and to avoid any leakage of formulation. The actual thickness of skin used in each experiment was measured.

Stripped skin samples were obtained from the above-described abdominoplasty skin as follows. The required skin portion was marked before being dermatomed. The SC of the selected portion was removed with help of SCOTCH® book tape No. 845. The procedure was repeated (typically 10-30 times) to make sure that the SC was removed convincingly. The resulting stripped skin, thus obtained, was either used immediately or wrapped and frozen at −20° C. The stripped skin samples were then sliced and transferred onto diffusion cell disks as described above. The actual thickness of skin used in each experiment was measured.

When additional intact and stripped skin samples were needed, frozen intact and stripped skin samples were thawed at room temperature, and the required tissue portions were then sliced and transferred onto diffusion cell disks as described above.

A Δ⁹-tetrahydrocannabinol formulation was prepared as follows. Suitable aliquots of Δ⁹tetrahydrocannabinol in 95% absolute ethanol (approximately 0.04 parts ethanol/ml) were transferred to a mixture of propylene glycol:water (90:10) and mixed well to obtain 8.59 mg/ml of drug concentration in each sample.

The in vitro experiments were carried out under the following conditions. The receiver solutions were maintained at 37° C. for 30 min prior to conditioning the diffusion cell lines. After cleaning the transfer tubes with 50% methanol for 1 h, the diffusion lines were conditioned by pumping the receiver solution through them for at least for 1 h. The diffusion cell mounting table (an aluminum block holder) was conditioned to keep the skin surface at 32° C. by circulating a water bath maintained at 37° C. Thereafter, the skin diffusion cell disks were transferred to the mounting table, and the skin diffusion cell disks were equilibrated by circulating a water bath maintained at 37° C. for 30 min. The donor cell was loaded with 240 μl of the Δ⁹tetrahydrocannabinol formulation and was covered with a suitable cap. The receiver solution was pumped through the receiver cell at a flow rate of 1.5 ml/hr for either 48 h or 96 h. Samples were collected using a fraction collector at 6 h intervals for either 48 h or 98 h, depending on the study. At the end of each experiment, the tissue was removed from the respective diffusion cell disk. Both the epidermal and dermal surfaces of the removed tissue sample was briefly washed with distilled water, and excess water was removed with blotting paper. The formulation covered skin surface of the washed and blotted removed tissue sample was stripped once with SCOTCH book tape No. 845 to remove the formulation, and the stripped, washed, and blotted removed tissue sample was sliced to smaller portions. The smaller portions were then transferred to a previously weighed scintillation vial, the combined weight of the scintillation vials and tissue was measured, and, from this, the weight of the tissue was calculated. The tissue samples were then digested overnight in 10 ml ACN on a shaker bath to estimate the drug penetration into skin layers.

BRIJ 98 pretreatment studies were carried out as follows. At the end of regular 48 h diffusion experiment, the samples collected in BRIJ 98 receiver solution were immediately removed and, without interruption of the experiment, replaced with BSA 4% receiver solution. The experiment was continued for a further 48 h period against both BRIJ 98 and BSA 4% controls.

The diffusion samples collected using either 4% or 6% BSA receiver solution were extracted in the following manner. To each sample was added 4-fold ACN, and the resulting mixture was vortexed for 1 min, then sonicated for 15 min, then vortexed again for 1 min, and finally centrifuged at 9000 rpm for 15 min. The supernatant was collected and transferred to silanized HPLC autosampler vials. 100 μl of each sample was injected for each HPLC run, and the recovery was 90%.

The diffusion samples collected using either 0.5% or 6% BRIJ 98 receiver solution were either injected directly into the HPLC apparatus or diluted 1:3 with ACN, vortexed for 1 min, and then injected into the HPLC apparatus. The injection volume was 100 μl, and recovery was 100%.

The diffusion samples were estimated using the following HPLC method. A mobile phase containing 80:20 ACN:phosphate buffer (25 mM KH₂PO₄+0.1% TEA, pH 3.0), a reverse phase C₈ column (BROWNLEE®, 220×4.6 mm, Spheri-5), and a guard column (BROWNLEE®, reverse phase, C₈, 15×3.2 mm, 7 μm particle size) were employed. The flow rate was 1.5 ml/min. Run time was 7.0 min, except that all BRIJ 98 samples were run for additional 7.0 min after each run time at a flow rate of 2.0 ml/min to wash the column of BRIJ 98 peaks). The detection wavelength was set to 215 nm, retention time was 4.0+0.1 min, linearity was 25-1000 ng/ml, and sensitivity was 5 ng/ml.

The data were treated as follows. Permeability coefficients of Δ⁹-tetrahydrocannabinol were calculated using the steady state skin flux and the saturation solubility of the compound in the vehicle employed.

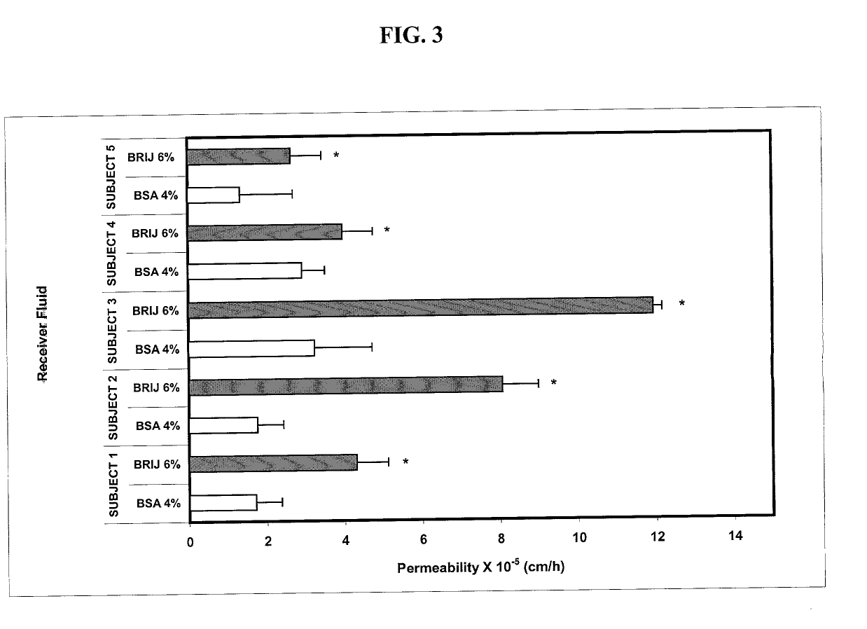

FIG. 3 shows the permeability of Δ⁹tetrahydrocannabinol through human skin in five subjects in presence of both BSA (4%) and BRIJ 98 (6%). It is evident from the data, that permeability of Δ⁹tetrahydrocannabinol is 2-5 fold higher in presence of BRIJ 98 solution relative to BSA solution, except in subject 4. These differences were statistically significant in all subjects (Student's t-test, p<0.05) indicated with asterisk in FIG. 3. No clear pattern between lag times was observed.

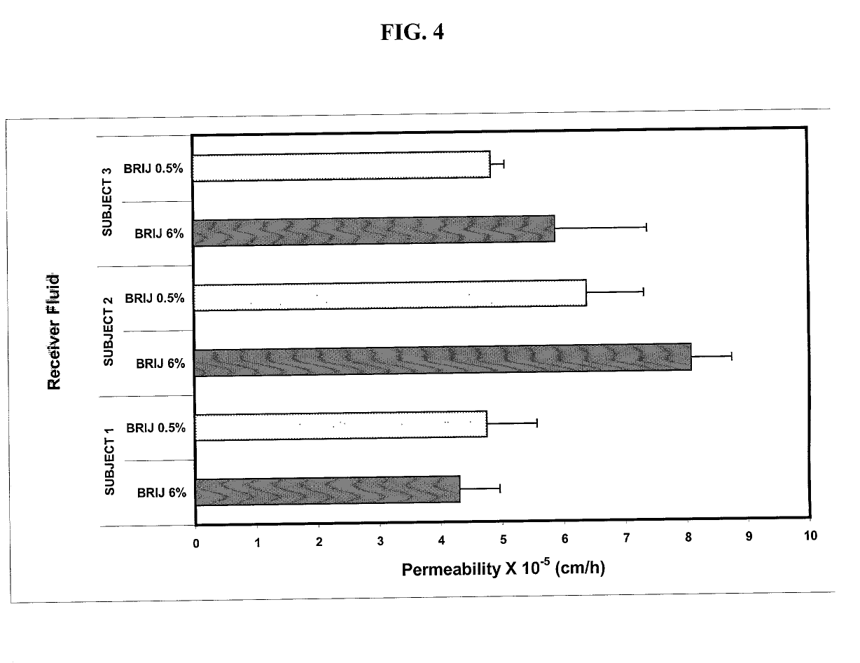

The higher permeability of Δ⁹tetrahydrocannabinol noted with BRIJ 98 (FIG. 3) could be due to possible damage to the skin caused by BRIJ 98, BRIJ 98 being a surfactant. Therefore, to minimize this possible damage to the skin and to enhance the solubility of drug, results using a low concentration of BRIJ 98 (0.5% solution) were compared against the results obtained using a 6% BRIJ 98 solution. The results are set forth in FIG. 4.

Interestingly, similar Δ⁹-tetrahydrocannabinol permeability results were obtained for the three subjects irrespective of whether 6% BRIJ 98 solution or 0.5% BRIJ 98 solution was employed, and whatever differences exist are not statistically significant (Student's t-test, p<0.05). No particular difference was observed between lag times. These findings demonstrate that one can minimize possible damage to skin without adversely affecting the solubility of drug in receiver solution by using a low (e.g., about 0.1 to about 5%, such as about 0.2 to about 4%, about 0.2 to about 3%, about 0.3 to about 3%, about 0.4 to about 3%, about 0.5 to about 3%, and/or about 0.5 to about 5%) concentration of BRIJ 98.

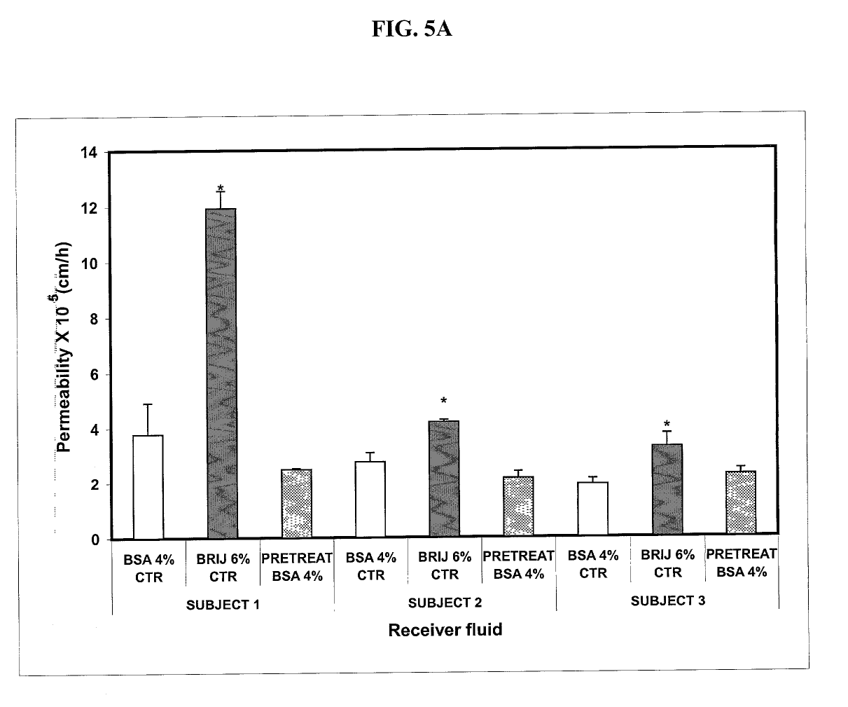

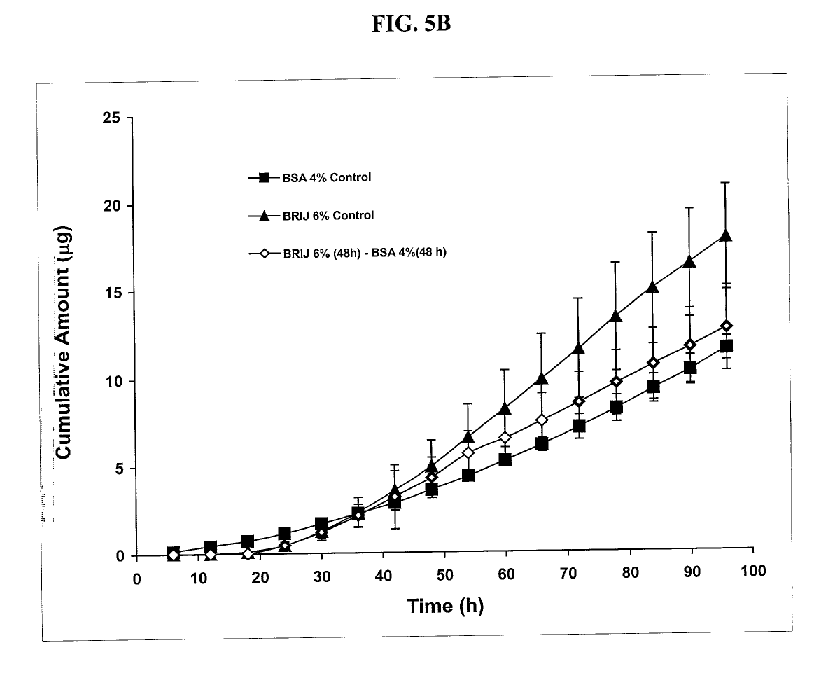

Experiments were conducted in which, at the end of BRIJ 98 pretreatment period (48 h), the receiver solution was changed to HHBSS/BSA (4%). If BRIJ was causing damage to the skin sample, this pretreatment step should also result in increased drug permeation in presence of HHBSS/BSA. The results of pretreatment studies performed to understand the possible damage effects of 6% BRIJ 98 in three subjects are shown in FIG. 5A. The Δ⁹-tetrahydrocannabinol permeability obtained between BRIJ and BSA solutions demonstrated once again that 6% BRIJ 98 resulted in higher drug permeation through human skin relative to BSA pretreatment and BSA control. Tissues pretreated with 6% BRIJ 98 for 48 h followed by a change to BSA (4%) for an additional 48 h exhibited permeability values similar to those of 4% BSA control after 96 h. In addition, referring now to FIG. 5B, there is no noticeable deviation from steady state in any of the permeability profile of 6% BRIJ 98 solutions during 96 h, while, in the pretreated samples, the profile tends to move closer and parallel to the 4% BSA control. No lag time differences between the receiver solutions were observed. From the above discussion and the data presented in FIGS. 5A and 5B, it is clear that BRIJ does not cause any damage to the skin. The one-way ANOVA and Toukai test comparison of above data suggest that there is significant difference between the permeability values of BRIJ 98 and 4% BSA solutions (this significance being noted in FIG. 5a with an asterisk) and that there is no significant difference between BSA control and pretreated/BSA solutions.

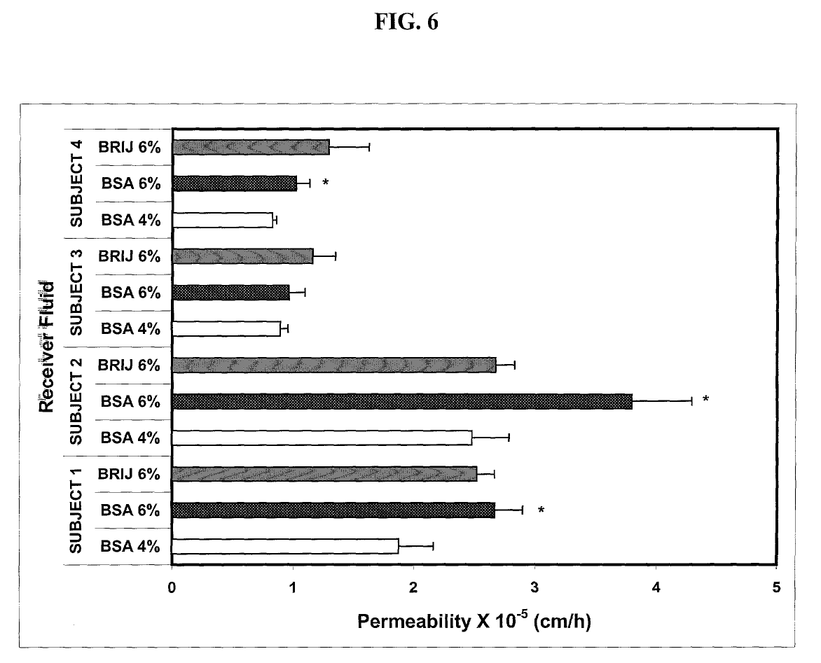

As discussed above, it is known that many drugs are metabolized during their permeation across human skin. To understand this metabolic process for cannabinoids and to thus be able to choose those cannabinoids that are likely to be active upon systemic absorption, the receiver solution should be such that it not only has appreciable solubility for the drug but also such that it does not adversely impact on the tissue's viability. In this context, HHBSS/BSA, being closer in composition to systemic fluids especially blood, can be considered a better receiver solution. Accordingly, experiments were conducted using 6% BRIJ 98, 4% BSA, and 6% BSA. The results obtained from this study in four subjects are shown in FIG. 6. It is clear from the data presented in FIG. 4 that 6% BSA has enhanced Δ⁹tetrahydrocannabinol permeability in all subjects by some 20-50% relative to 4% BSA (except in subject 3). Additionally, in three out of four subjects in FIG. 6, the permeability difference between 6% BSA and 4% BSA was considered to be statistically significant (Student's t-test, p<0.05). No lag time differences between receiver solutions were observed.

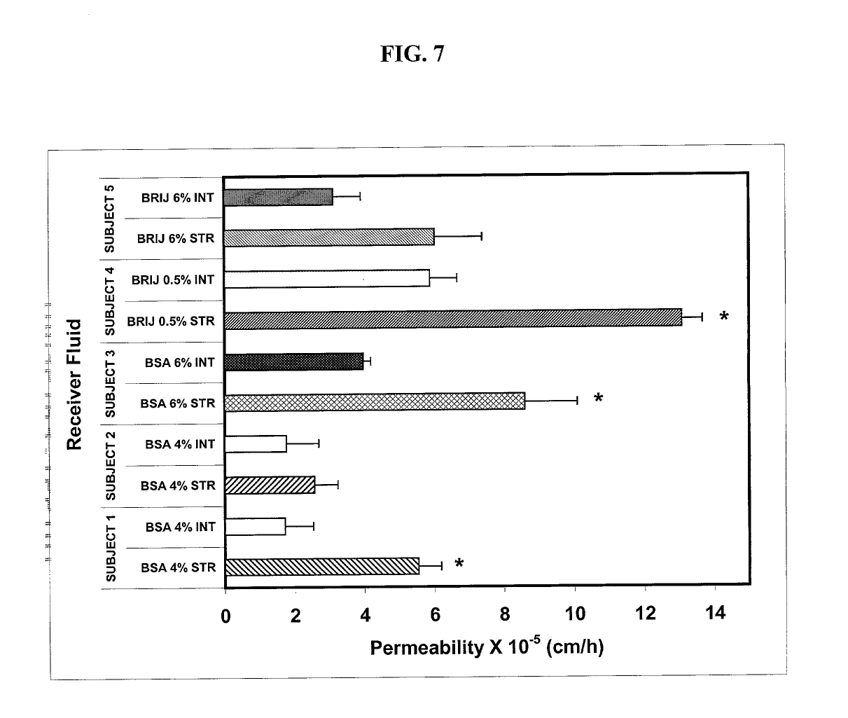

It is conventional practice to perform tape strip studies against intact skin to understand clearly the barrier effects of SC. Thus, tape strip studies for Δ⁹-tetrahydrocannabinol against intact skin were conducted using the above-identified receiver solutions. The results obtained in various subjects are shown in FIG. 7. It is clear from FIG. 7 that the permeability values of tape stripped skin are 2-3 times higher relative to their intact skin counterparts, and, in the case of 6% BRIJ 98 receiver solution, a six fold difference was observed. Moreover, the observed mean lag times were found to be shorter for stripped skin (7.8+3.9 h) relative to intact skin (18.0+3.4 h). The Student's t-test (p<0.05) comparison of data suggests that the permeability differences were significant in three out of five subjects. This demonstrates that the SC acts as barrier for permeation of Δ⁹-tetrahydrocannabinol molecule.

### Example 3

### Delivery of 2-[3-methyl-6-(1-methylethenyl)2-cyclohexen-1-yl]-5-pentyl-1,3-benzenediol Across Skin

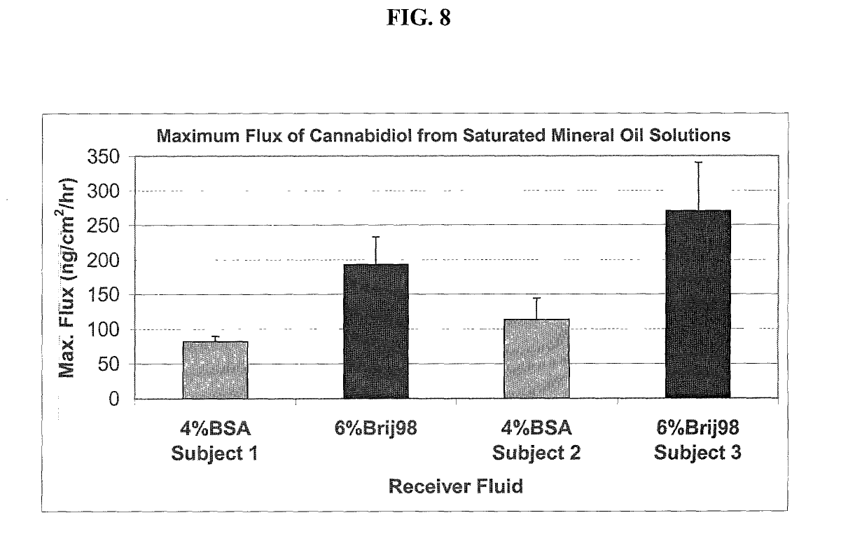

The flux of 2-[3-methyl-6-(1-methylethenyl)-2-cyclohexen-1-yl]-5-pentyl-1,3-benzenediol (a cannabidiol) (“CBD”) across human skin was measured following the procedures set forth in Example 2, except that the donor compartment was charged with CBD in mineral oil (saturated) instead of Example 2's Δ⁹-tetrahydrocannabinol in propylene glycol (50 mg/ml). Two receiver solutions were employed: 4% BSA and 6% BRIJ 98. The experiments were run as in Example 2, and the maximum flux was calculated. The results are presented in FIG. 8.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed is:

1. A method for relieving symptoms associated with illness or discomfort associated with the treatment of illness in a subject, said method comprising:
providing a cannabinoid composition comprising at least one cannabinoid selected from the group consisting of Δ⁹-THC, cannabinol, cannabidiol, nabilone, levonantradol, (−)-HU-210, +)-HU-210, 11-hydroxy-Δ⁹-THC, Δ⁸-THC-11-oic acid, CP 55,940, and R(+)-WIN 55,212-2; and
delivering the cannabinoid transdermally to the subject.

2. A method according to claim 1, wherein said delivering comprises:
providing an occlusive body which comprises the cannabinoid; and
positioning the occlusive body on the subject's skin under conditions effective to transdermally deliver the selected cannabinoid to the subject's skin.

3. A method according to claim 2, wherein the occlusive body further comprises:
an impermeable backing; and
a rate-controlling microporous membrane, wherein the backing and membrane define a cavity therebetween and wherein the selected cannabinoid is disposed within the cavity

4. A method according to claim 3, wherein the selected occlusive body further comprises:
a viscous flowable gel disposed within the cavity, wherein the viscous flowable gel immobilizes the cannabinoid within the cavity.

5. A method according to claim 3, wherein the occlusive body further comprises:
an adhesive for attaching the occlusive body to skin.

6. A method according to claim 1, wherein the illness is selected from the group consisting of AIDS and cancer.

7. A method according to claim 1, wherein the cannabinoid composition comprises two or more cannabinoids selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2 and wherein each of the two or more cannabinoids is delivered transdermally to the subject.

8. A method according to claim 1, wherein the selected cannabinoid is delivered via a topical formulation.

9. A method according to claim 1, wherein the selected cannabinoid is delivered via a patch.

10. A method according to claim 1, further comprising the steps of:
providing an opiate; and
delivering the opiate transdermally with the selected cannabinoid to the subject.

11. A method according to claim 1, wherein the cannabinoid composition comprises Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

12. An occlusive body comprising:
an impermeable backing;
a rate-controlling microporous membrane, wherein said backing and said membrane define a cavity therebetween; and
a cannabinoid disposed within the cavity.

13. An occlusive body according to claim 12 further comprising:
a viscous flowable gel disposed within the cavity, wherein said viscous flowable gel immobilizes said cannabinoid within the cavity.

14. An occlusive body according to claim 12, wherein said cannabinoid is selected from the group consisting of Δ⁹-THC, Δ⁸-THC, cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55,212-2, and combinations thereof.

15. An occlusive body according to claim 12 further comprising:
an adhesive for attaching said occlusive body to skin.

16. An occlusive body according to claim 12, wherein said membrane has an exterior surface coated with an adhesive.

17. An occlusive body according to claim 16, wherein the adhesive is a silicone-based adhesive.

18. An occlusive body according to claim 12, wherein said membrane is hydrophobic and wherein said occlusive body further comprises:
a hydrophilic wetting agent disposed in the cavity.

19. An occlusive body according to claim 12, wherein said occlusive body further comprises:
water and a surfactant, wherein said water and surfactant are disposed in the cavity and wherein said surfactant is selected from a viscosity modifier and a gelling agent.

20. An occlusive body according to claim 19, wherein said surfactant comprises methyl cellulose.

21. An occlusive body according to claim 12 further comprising:
an opiate, wherein said opiate is disposed in the cavity.

22. An occlusive body according to claim 12, wherein said cannabinoid is a combination of cannabinoids comprising Δ⁹-THC, Δ⁸-THC, cannabinol, cannabidiol, nabilone, levonantradol, (−)-HU-210, (+)-HU-210, 11-hydroxy-Δ⁹-THC, Δ⁸-THC-11-oic acid, CP 55,940, R(+)-WIN 55,212-2, and combinations thereof.

23. A method for increasing the concentration of cannabinoids or cannabinoid metabolites in a subject, said method comprising:
contacting the subject's skin with a compound selected from the group consisting of Δ⁹-THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

24. A method according to claim 23 further comprising:
contacting the subject's skin with a permeation enhancer.

25. A method according to claim 23 further comprising:
contacting the subject's skin with a cannabinoid metabolism inhibitor.

26. A method according to claim 23, wherein the compound is a combination of compounds comprising Δ⁹THC, cannabinol, cannabidiol, nabilone, levonantradol, (−)-HU-210, (+)-HU-210, 11-hydroxy-Δ⁹-THC, Δ⁸-THC-11-oic acid, CP 55,940, and R(+)-WIN 55,212-2.

27. A method for assessing the permeability of skin to a cannabinoid, said method comprising:
providing a skin sample, said skin sample having a first surface and an opposing second surface;
providing a donor solution comprising a cannabinoid;
providing a receiver solution comprising from 0.1 to 5% of a polyoxyethylene oleyl ether;
disposing the skin sample between the donor solution and the receiver solution such that the skin sample separates the donor solution and the receiver solution, such that the donor solution is in contact with the skin sample's first surface, and such that the receiver solution is in contact with the skin sample's second surface; and
detecting cannabinoid present in the receiver solution.

29. A method according to claim 27, wherein the receiver solution comprises about 0.5% of a polyoxyethylene oleyl ether.
